(19) United States
(12) Patent Application Publication
(10) Pub. No.: US 2005/0245824 A1
(43) Pub. Date: Nov. 3, 2005

(54) High-intensity focused-ultrasound hydrophone

(75) Inventors: Mark E. Schafer, Ambler, PA (US); G. Wayne Moore, Lyons, CO (US); James M. Gessert, Loveland, CO (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Acoustic Marketing Research, a Colorado corporation, d/b/a Sonora Medical Systems, Inc., Longmont, CO (US)

(21) Appl. No.: 11/111,476

(22) Filed: Apr. 20, 2005

Related U.S. Application Data
(60) Provisional application No. 60/564,005, filed on Apr. 20, 2004.

(51) Int. Cl. A61B 8/14

(52) U.S. Cl. 600/442

(57) ABSTRACT

Methods and systems are disclosed for providing a self contained hydrophone for measuring characteristics of HIFU fields. HIFU field characteristics are measured using transducer elements receiving reflected and or scattered HIFU fields scattered from an integrated scattering device and providing for use of more responsive piezoelectric materials. The transducer element and/or elements are configured in the integrated hydrophone to provide for in-phase reception of HIFU waves scattered from the scattering device. A positioning mechanism may be used that with the use of the transducer element and/or transducer elements as pulse-echo devices may provide for tuning of the integrated HIFU hydrophone. Further, the integrated structure of the hydrophone may be sealed to provide for use of cavitation mitigating liquids.

### CROSS-REFERENCES TO RELATED APPLICATIONS

This application is a nonprovisional of and claims the benefit of the filing date of U.S. Provisional Patent Application Ser. No. 60/564,005, filed Apr. 20, 2004, entitled, HIGH-INTENSITY FOCUSED-ULTRASOUND HYDROPHONE, the complete disclosure of which is incorporated herein by reference for all purposes.

### BACKGROUND OF THE INVENTION

The present invention relates generally to ultrasonics and the measurement of ultrasonic fields. More specifically, embodiments of the present invention relate to methods and systems for measuring high intensity focused ultrasound (“HIFU”) fields.

HIFU fields and devices capable of producing HIFU fields are currently being used and developed for the destruction of cancerous tumors or other unwanted tissue structures within the body. In HIFU medical treatments, HIFU fields are narrowly focused to provide that ultrasonic energy is directed only at a specific targeted region of tissue within the patient. The focusing in HIFU devices may direct ultrasonic energy to an area the size of a grain of rice. Outside of the focused region, the energy of the ultrasonic field produced by the HIFU medical devices is insufficient to cause tissue damage. Consequently, the ultrasonic fields produced by a HIFU medical device may pass through non-targeted tissue without causing damage before arriving at a targeted tissue location in the patient. Because of the tight focusing of an ultrasound beam and the energy of the acoustic waves used in HIFU devices, the ultrasonic fields produced at the point of focus are of sufficiently high intensity to raise the temperature of the targeted tissue above 45° C., the point at which proteins within the tissue denature and the cells within the tissue die.

To understand HIFU devices for patient treatment, experimental and/or regulatory reasons, it is necessary to measure HIFU dosage, calibrate HIFU output, measure output characteristics, and evaluate the effects of the ultrasonic fields for different field strengths. To do this, it is necessary to accurately measure the HIFU fields produced by the HIFU devices. To fully understand the HIFU fields, the fields must be quantified with respect to their distribution in space (spatial measurement), their extent in time (temporal measurement), and their frequency content. Analysis of frequency data is necessary due to the non-linear nature of wave propagation within tissue. Devices that are used to measure ultrasonic fields are called hydrophones.

A problem with the measurement of HIFU fields using conventional hydrophones, however, is that the ultrasound fields of most interest, the ones that are sufficiently intense to denature proteins and/or cause necrosis, are so powerful that they may often destroy or significantly alter the properties of the hydrophone. Further, to effectively measure ultrasound fields from HIFU devices with medical applications, the measurement device must be able: (a) to repeatedly measure ultrasound fields over a wide frequency range with a relatively flat frequency response—from 500 kHz to 20 MHz, when harmonics are included—and (b) to measure small field areas, that may be less than 0.5 mm×0.5 mm, where the ultrasounds fields have very high focused intensities, often over 500 W/cm². Hydrophones that are capable of performing such measurement functions are often unable to cope with higher energy ultrasonic fields, such as HIFU fields.

### BRIEF SUMMARY OF THE INVENTION

Embodiments of the present invention thus provide a self contained hydrophone using a shell structure with at least one opening to provide for access of HIFU ultrasound waves, wherein one or more transducers may be attached to the shell structure to measure HIFU ultrasound waves scattered and/or reflected by a scattering/reflecting point on a scattering/reflecting object where the scattering/reflecting point is located within the shell structure. In embodiments of the present invention, the scattering and/or reflecting of the HIFU ultrasound fields onto the transducer may provide for mitigation of the damage to the transducer caused by the HIFU ultrasound fields and may provide for repeated use of the transducer. In embodiments of the present invention where more than one transducer is used in the hydrophone, the transducers may be arranged relative to the scattering point to provide for combination of the outputs of the transducers so as to increase the signal to noise ratio. In other aspects, a large area of an inner surface of the shell structure may be disposed with a film of a piezoelectric polymer to provide for wide bandwidth response and strong signal output.

In an embodiment of the present invention, the shell structure may be provided with acoustic windows on the one or more openings to create a sealed space into which a cavitation mitigating liquid may be introduced to reduce cavitation effects associated with the scattering object and/or the transducer(s) and/or the propagation of nonlinear waves in the hydrophone. In certain embodiments, the transducer(s) may be driven to provide a pulse signal that may be scattered/reflected from the scattering/reflective point and pulse-echo characteristics of the pulse signal may be measured to locate a preferred location for the scattering/reflective point relative to the transducers. In certain aspects, the scattering/reflective point may be moveable in three-dimensions within the shell structure to provide for a self-contained unit that may be simply and effectively tuned by an operator.

### BRIEF DESCRIPTION OF THE DRAWINGS

A further understanding of the nature and advantages of the present invention may be realized by reference to the remaining portions of the specification and the drawings wherein like reference numerals are used throughout the several drawings to refer to similar components.

FIG. 1A provides a schematic illustration of a cross-view of a high-intensity focused-ultrasound hydrophone in accordance with an embodiment of the present invention;

FIG. 1B provides a top view of a transducer arrangement used within a high-intensity focused-ultrasound hydrophone in accordance with an embodiment of the invention;

FIG. 2A provides a block diagram illustrating a pulser circuit that may be used to drive a transducer to produce ultrasound waves in combination with an isolation device to isolate driving pulses from signals received by the transducer in accordance with an embodiment of the invention;

FIG. 2B illustrates the operation of a transducer as an ultrasound pulse-echo transmitter in accordance with an embodiment of the present invention; and

FIG. 3 provides a flow diagram providing an overview of the functional hierarchy used in an embodiment of the present invention.

### DETAILED DESCRIPTION OF THE INVENTION

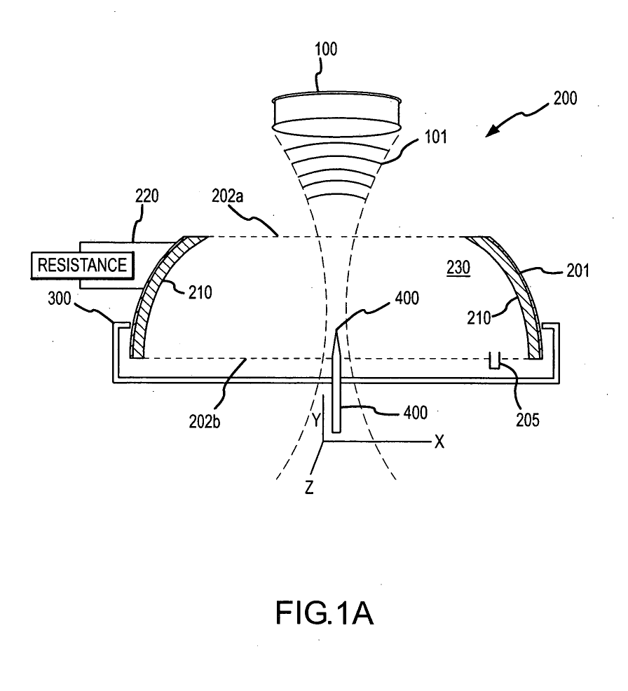

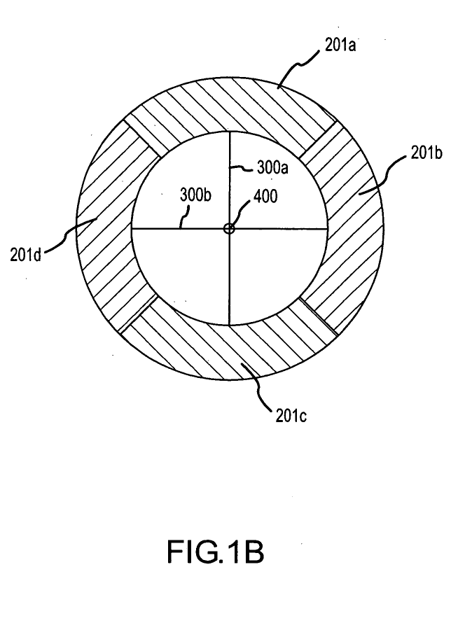

Embodiments of the present invention are directed generally to hydrophones that may be used to measure high-intensity focused ultrasound fields. FIGS. 1A and 1B provide a general structural overview of such a system that may be configured according to an embodiment of the invention appropriate for measuring high-intensity focused ultrasound fields, particularly, but not by way of limitation, for calibrating and testing high-intensity focused ultrasound fields from medical devices.

While much of the discussion below specifically discusses apparatus and methods that are suitable for measuring HIFU fields with certain properties, this is intended merely for exemplary purposes and the invention is not intended to be limited by the operational frequency characteristics or intensities described. As illustrated in further detail below, embodiments of the present invention include one or more “transducer elements” and/or “transducers” which refer to elements adapted to convert energy form one form to another that may be configured with electrical circuits to transmit acoustic radiation and/or to receive acoustic radiation. While such elements are referred to generically herein as “transducer elements” and/or transducers, reference is sometimes also made herein to “receiver elements” and/or “receivers” and to “transmitter elements” and/or “transmitters” to distinguish them on the basis of their function.

For illustrative convenience, figures may illustrate operation of the present invention in which one or two transducer elements are illustrated and may include operation and/or associated components of these one or two transducer elements. However, embodiments of the present invention may involve the use of more than two transducer elements and require additional components associated with these transducer elements. Hence, the figures and associated descriptions should not be considered to limit the present invention to the use of two or less transducer elements.

To measure properties of HIFU fields, it may be necessary to use a transducer element to convert the acoustic energy of the HIFU field to another form that may be easily processed and quantified. Piezoelectric materials may be used as transducers to convert the acoustic energy of the HIFU fields to electrical energy. Piezopolymer materials, such as polyvinylidene difluoride (PVDF), have transducer properties that are suitable to provide the bandwidth and linearity requirements necessary for measuring ultrasonic fields produced by HIFU devices. These materials, however, are relatively fragile and may literally melt or otherwise become damaged at the intensity levels produced by the HIFU devices, especially at when position in close proximity to the focal point of such devices. Piezoceramic materials, such as a lead zirconate titanate (PZT), on the other hand, are more physically robust than piezopolymer materials, however, piezoceramic materials are not able to provide the linearity of response necessary to effectively measure the characteristics of HIFU fields at all intensity levels.

Various references discuss and describe hydrophones for measuring HIFU fields. However, these references disclose devices that are not user friendly, that do not fully integrate necessary components, that utilize transducer elements that may be destroyed by the more powerful HIFU fields, that are not be capable of providing wide bandwidth response, that are difficult to “tune” for maximal response, that have low signal to noise capabilities and/or that do not mitigate cavitation effects. Thus, a need in the art exists for a self contained, user friendly hydrophone that may be easily tuned and can provide wide bandwidth capabilities, linear response, high signal to noise ratios, mitigate cavitation and/or is capable of repeated use without degradation.

FIG. 1A provides a schematic illustration of a cross-view of a high-intensity focused-ultrasound hydrophone in accordance with an embodiment of the present invention. In accordance with an embodiment of the present invention, a completely integrated HIFU hydrophone 200 is depicted for measuring HIFU fields. As illustrated, HIFU source 100 is a HIFU transmitting device the output characteristics of which are to be tested by the HIFU hydrophone 200. As illustrated, the HIFU hydrophone 200 is aligned to receive a HIFU beam 101 radiated from the HIFU source 100. Merely by way of example, HIFU transmitters for use as medical devices may transmit HIFU fields comprising waveforms including multiple cycles of a fundamental frequency, typically in the range from 500 kHz to 20 MHz. The dashed lines indicate the extent of the HIFU ultrasound field in cross section. For operational purposes, the HIFU source 100 may tightly focus the HIFU beam onto a target. As such, for testing and/or measuring purposes, it may be necessary to determine the characteristics of such a tightly focused HIFU beam. As persons of skill in the art may appreciate, as the HIFU beam narrows, the local intensity, which is measured in units of watts per square centimeter (W/cm²) rises. As such, the area of interest for analysis purposes, the focal point and proximal locations, receive high intensity acoustic radiation.

In the illustrated embodiment, the components of the HIFU hydrophone 200 are supported by a shell structure 201. The shell structure 201 may be a generally rigid structure that may be capable of integrating the components of the HIFU hydrophone 200 into a unitary user friendly device, may hold the components of the HIFU hydrophone 200 in a preferred alignment and may provide for tuning of the HIFU hydrophone 200. The shell structure may have an inner surface defining an inner volume and the inner surface may be used to support elements for receiving and measuring HIFU fields. In certain embodiments, the shell structure 201 may be made from material that is durable enough for measuring HIFU fields and the use of the HIFU hydrophone 200 in the field and withstanding the effects of the direct and/or reflected impact of HIFU beams.

In some embodiments of the present invention, the shell structure 201 may be curved in shape. Further, as illustrated, the shell structure 201 may be spherically curved in shape about a spherical center. However, in practice, it is often difficult to manufacture perfectly curved and/or spherically curved shell structures. As such, shell structures that are substantially spherically curved may be used in some embodiments of the present invention and cylindrical shell structures may be used in other embodiments. In still other embodiments of the present invention, the shell support may be polygonal in shape. In some embodiments utilizing a polygonal shell structure, desired ultrasound reception properties of the HIFU hydrophone 200 may be obtained by orienting and/or positioning one or more ultrasound receivers inside the polygonal shell structure, as described in more detail below, to be equidistant from a scattering point and/or to used curved and/or spherically curved transducer elements in the polygonal shell structure.

The shell structure 201 may be shaped as a doubly truncated sphere, which is defined as a sphere intersected by two planes, so that the shell structure is spherically curved about a center point. The truncated ends of the sphere may be parallel to one another or may be otherwise oriented and the truncated ends may be positioned at different distance from the center point of the spherically curved shell structure provided that they are positioned so that the center point of the shell structure 201 lies within an interior area defined by the doubly truncated sphere.

Membranes 202a and 202b are barriers that may be used to seal the HIFU hydrophone 200. By sealing the HIFU hydrophone 200, the shell structure 201 and the membranes 202a and 202b form an interior volume 230. The membranes 202a and 202b may be acoustically transparent to ultrasound waves with frequencies in the operating range of the HIFU source, which for a medical HIFU source includes ultrasound waves with frequencies approximately in the range of 500 KHz to 20 MHz. Using acoustically transparent membranes provides that the membranes are not damaged by and do not interfere with the HIFU beam 101.

In certain embodiments, the membranes 202a and 202b may be impermeable to provide that the interior volume 230 may be filled with a liquid. Merely by way of example, the membranes 202a and 202b may be made from polyethylene a substance that is both impermeable and transparent to HIFU acoustic waves at the relevant acoustic frequencies. In certain aspects, the interior volume 230 may be filled with or emptied of liquid via an inlet/outlet mechanism 205. In this way, the interior volume 230 may be filled with a liquid that mimics the acoustic transmission properties of tissue, wherein the tissue mimicking liquid has similar propagation speed, attenuation, and nonlinearity response as soft tissue. Use of a tissue mimicking liquid may provide for an analysis of the characteristics of the HIFU beam 101 in tissue like conditions. In other aspects, the interior volume may be filled with a liquid, such as glycerin, that may reduce cavitation effects produced by the HIFU beam 101 and/or nonlinearity of the HIFU beam 101. Mitigation of cavitation effects using a suitable liquid may help to maintain longevity of the components of the HIFU hydrophone 200, including the longevity of the transducer and the scatterer. A liquid that provides a balance of mimicking tissue properties and mitigating cavitation may be desirable for use in the interior volume 230. As persons of skill in the art may appreciate, tissue mimicking liquids for ultrasound purposes are known and, therefore, are not listed here.

In certain aspects, the single transducer element 210 may be divided into a plurality of functionally separate transducer elements. In alternative embodiments, a single transducer element that is smaller than the interior surface of the shell structure 201 may be supported by the shell structure 201. In further embodiments, a plurality of discrete transducer elements may be supported by the shell structure 201. In embodiments in which the interior surface is substantially covered with the transducer element 210 and/or multiple transducer elements are supported in the shell structure 201, the transducer element(s) may provide a large surface area for receiving the scattered/reflected HIFU beam and, as a result, may produce strong signal strength.

In the illustrated embodiment, an interior surface of the shell structure 201 is shown coated with and/or otherwise supporting a transducer element 210. The transducer element 210 is formed from a material having piezoelectric properties, i.e., deformation of the piezoelectric material results in a change in electric field. Alternatively, the piezoelectric material may be used as a transmission element by imposing a periodic electric field to produce acoustic waves from the resulting periodic deformation of the piezoelectric material.

The transducer element 210 may comprise any piezoelectric material responsive to HIFU ultrasound waves. In certain embodiments the transducer element 210 may comprise a piezopolymer material, such as polyvinylidine difluoride (PVDF), one of its copolymers, or the like. Piezopolymer materials have been demonstrated to be effective as hydrophone transducers material for ultrasonic frequencies from 500 kHz to 50 MHz. In certain aspects the piezoelectric material may be a film or the like that is coated onto the interior surface. In other aspects, the piezoelectric material may comprise an individual transducer element one or more of which may be supported by the shell structure 201. Electrodes may be sputtered onto the piezoelectric film and may provide for electrical coupling with and in electrical circuits associated with the HIFU hydrophone. In still further aspects, a film of the piezopolymer material may be disposed between two other conductive films and the combination of the three films may be used as a transducer element and coupled with the shell structure 201, where the use of the conductive films may provide for the elimination of the need of any electrode in direct contact with the piezopolymer. In some embodiments, a monitoring circuit 220 may be coupled with the transducer element 210 to monitor physical characteristics of the transducer element 210. Physical characteristics of the transducer element may be monitored to ascertain when a new transducer element should be used and/or to calibrate outputs from the transducer element 210. Physical properties monitored using the monitoring circuit 220 may include resistance across the transducer element 210.

A reflective scatterer 400 may be positioned in the HIFU hydrophone 200 so that at least a portion of the reflective scatterer 400 is located in the interior volume 230. The reflective scatterer 400 may be aligned within the HIFU hydrophone 200 so that the HIFU beam 101 may be broadly scattered and/or reflected inside the HIFU hydrophone 200. As such, the HIFU fields scattered/reflected by the reflective scatterer 400 may be readily detected in the HIFU hydrophone 200 by the transducer element 210. Embodiments of the invention, therefore, provide an integrated device that advantageously includes a scattering element with a remote transducer element so that the temperature effects at the focus of the HIFU beam are not applied in totality to the transducer element and, as a consequence, do not alter or destroy the transducer element.

To provide for strong scattering/reflecting of the HIFU beam 101, the reflective scatterer 400 may be rigidly supported and a portion of the reflective scatterer 400 positioned directly in the path of the HIFU beam 101 may have dimensions smaller than the wavelength of the HIFU beam 101. In certain aspects, the reflective scatterer 400 may be formed from metal and/or glass and the reflective scatterer 400 may be tapered and/or shaped so as to have a scattering end 401 with preferred proportions relative to the wavelength of the HIFU beam 101 that may be used as the active area of the reflective scatterer 400 for reflecting/scattering the HIFU beam 101. The scattering end 401 may be sized in the general region of a tenth to a hundredth of a millimeter to provide for strong and/or broad scattering/reflecting of the HIFU beam 101. In some embodiments, a glass fiber-optic fiber may be used as the reflective scatterer 400. In such embodiments, a tip of the fiber optic may be used as the scattering end 401. The tip of the fiber optic may provide for strong reflection/scattering of the HIFU fields and may be shaped, tapered, and/or the like to provide for desired reflection/scattering. Because of the flexibility of such fiber optic fibers, support mechanisms may be used to support the fiber optic. Merely by way of example, a capillary tube may be used to support the fiber optic to provide for positioning the fiber optic and/or the like.

In some embodiments, the shell structure 201 may be used without the membranes 202a and 202b. In such embodiments, the shell structure 201 may be used to support the transducer element 210 relative to the scattering end 401 and the HIFU fields may enter the shell structure through an opening and may be scattered/reflected from the scattering end 401 onto the supported transducer element 210. As with embodiments using the membranes 202a and 202b, the shell structure 201 may be shaped as a truncated or double truncated sphere where the truncated end(s) of the sphere may be openings through which the HIFU fields may pass, etc. By positioning the scattering end 401 at a center of curvature and/or a center of the shell structure 201, reception of reflected/scattered HIFU waves may occur in phase. Further, by utilizing large areas of the interior of the shell structure 201 to support a plurality of the transducer elements 210 or a film of the transducer element 210, strong signal reception may be achieved. In some embodiments, the shell structure 201 may be configured with the transducer element 210, the reflective scatterer 400 and/or a mechanism for moving the reflective scatterer 400 to provide an integrated HIFU hydrophone capable of using a piezopolymer.

As discussed above, in certain embodiments of the present invention, cavitation mitigating liquids may be provided in the interior volume 230. In certain aspects, the inlet/outlet mechanism 205 may be used to introduce and remove liquids from and to the inner volume 230. If desired by the user, the inlet/outlet mechanism 205 may be used to provide for different fluids surrounding the scatterer depending upon the HIFU beam analysis to be performed. The use of a cavitation mitigating liquid in the interior volume 230 may also provide for reduction in the effect of cavitation noise on the measurement of the HIFU field. In embodiments of the present invention, cavitation may be mitigated by the use of a substantially spherically curved shell structure 201 with the scattering end 401 located at a center of the spherically curved shell since cavitation occurs stochastically in the general region of the scatterer, cavitation noise is reduced because any cavitation bubble collapse, which does not occur exactly at the spherical focal point of the receiver will not produce a fully coincident signal and, therefore, its effect will be significantly reduced.

One example of a liquid that may provide for mitigation of cavitation by HIFU fields is glycerin. In embodiments employing a cavitation mitigating liquid, the cavitation mitigating liquid may be distributed so as to come into contact with the scattering end 401 to mitigate the effects of cavitation on the scattering end 401 and provide for increased longevity and/or effectiveness of the scattering end 401. Alternatively or in combination, the cavitation mitigating liquid may be distributed so as to be in contact with the transducer element 210 and may provide for increased longevity and/or effectiveness of the transducer element 210.

In an embodiment comprising a substantially spherically curved transducer element, the HIFU beam 101 may enter the hydrophone 200 and be scattered/reflected from the scattering end 401 of the reflective scatterer 400. The reflected/scattered waves radiate spherically from the scattering end 401 towards the transducer element 210. By placing the scattering end 401 at a symmetry point relative to the transducer element 210, the waves emanating from scattering end 401 all have substantially the same travel time to the transducer element 401. Consequently, the reflected/scattered waves may all be substantially in phase as they strike a surface of the transducer element 210 and may, as a result, cause a substantial reinforcement of the reflected/scattered waves received by the transducer element 210 because all portions of the waves received by the transducer element 210 may contribute to producing an electrical output from the transducer element 210.

While using the HIFU hydrophone 200 to measure the HIFU 101, in embodiments using multiple transducers, outputs from the multiple transducers may be combined into a single output waveform increasing the signal to noise ratio of the measurement. In embodiments utilizing a single transducer element, the wave reinforcement effect may be obtained by using a curved transducer element and the effect may be increased by using a curved transducer element with a large surface area. In non-spherical alignment configurations, a plurality of transducer elements may be cylindrically arranged around the scattering end 401 or positioned equidistant from the scattering end 401 to obtain a similar type effect. Embodiments of the invention provide a mechanism for combining outputs from different receiver elements into a single output. This is in contrast to systems that only combine signals from different electrode paths, all of which lead to a single receiver element.

A positioning mechanism 300 may be coupled to the shell structure 201 and the reflective scatterer 400 and may provide for movement of the reflective scatterer 400 in three dimensions inside the interior volume 230. In certain aspects, a more rudimentary configuration may be used that may only provide for the positioning mechanism 300 to move the reflective scatterer 400 in two-dimensions. To provide for alignment of the scattering end 401, the positioning mechanism 300 may be a rigid frame structure that holds the reflective scatterer 400 in place relative to the shell structure 200 and/or the transducer element 210, and maintains the scattering end 401 at the center point, center of curvature, center of symmetry, the equidistant position of the shell structure 200 and/or the transducer element 210.

In embodiments of the HIFU hydrophone 200, the scattering end 401 may be optimally positioned at the: (a) center point about which the shell structure and/or the transducer element 210 is spherically curved; (b) center of curvature of the shell structure; (c) symmetry center of a plurality of transducer elements; and/or (c) equidistant from each of the plurality of transducer elements. In an embodiment utilizing a single transducer element, response of the transducer element may be optimized by locating the reflective scattering end 401 at a center of curvature of the transducer element. In embodiments comprising more than one transducer element, positioning the scattering end 401 at a center point, center of curvature, center of symmetry, equidistant position, and or the like, of the one or more transducer elements may provide that the ultrasound waves reflected and/or scattered by the scattering end 401 may be received essentially in-phase by the transducer element(s) providing for increased signal reception and an increased signal to noise ratio.

FIG. 1B provides a top view of a transducer arrangement used within a high-intensity focused-ultrasound hydrophone in accordance with an embodiment of the invention. In the illustrated embodiment, the positioning mechanisms 300a and 300b are shown that may provide for the positioning/alignment of the reflective scatterer 400 in the HIFU hydrophone 200. A further positioning mechanism, not shown, may provide for the movement in an axis perpendicular to the illustration. In the illustrated embodiment, the transducer element 210 is shown separated into four separate receiver elements, 210a, 210b, 210c, and 210d. The separate transducer elements 210a, 210b, 210c, and 210d may be physically separated and/or electrically separated. Each of the transducer elements 210a, 210b, 210c, and 210d element may be electrically independent in some embodiments.

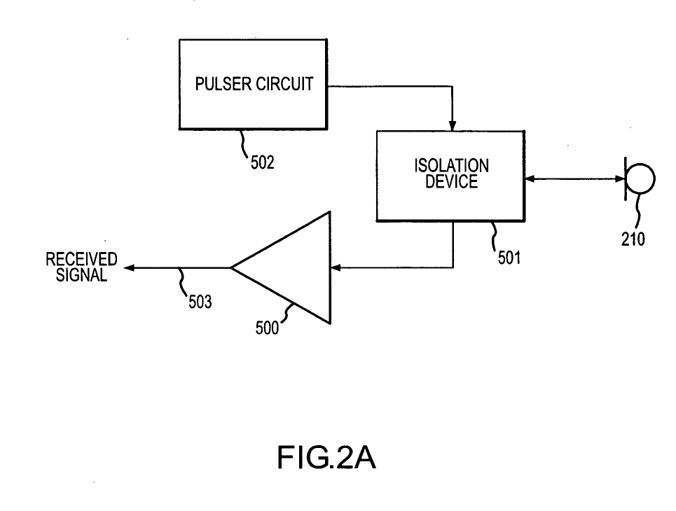

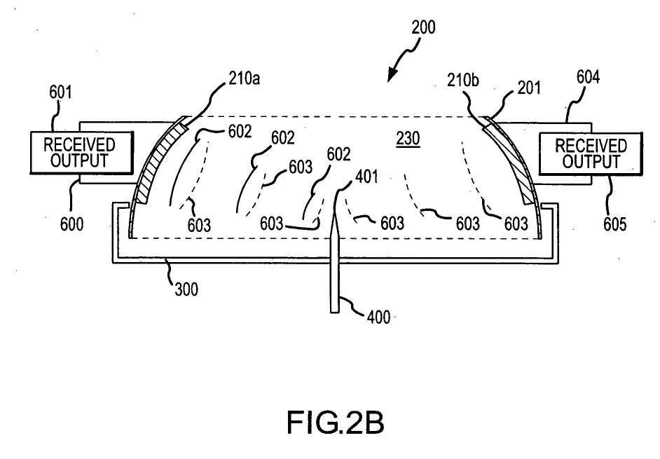

FIG. 2A provides a block diagram illustrating a pulser circuit that may be used to drive a transducer to produce ultrasound waves in combination with an isolation device to isolate driving pulses from signals received by the transducer in accordance with an embodiment of the invention. In the illustrated embodiment, a pulser circuit 502 may be used to pulse the transducer element 210. Applying an electrical pulse to the transducer element 210 may cause the transducer element 210 to periodically deform in response to the electrical pulse and create acoustic waves. As discussed with regard to FIG. 2B, using this effect, the transducer element 210 may be used as a transmitting device for purposes of positioning the reflective scatterer 400.

For purposes of reflective scatterer positioning, among other functions, the transducer element 210 may be used as an ultrasound pulse-echo transmitters, wherein it may operate as a receiving device at the same time it is performing as a transmitting device. To prevent interference and distortion of transmitted and/or received signals from the transducer element 210 an isolation device 501 may be used to electrically isolate hydrophone elements. In an embodiment of the invention, the isolation device 510 may be located between the pulser circuit 502 and a received signal amplifier 500, which may be used to amplify signals received by the transducer element 210, to isolate the received signal amplifier 500 from the pulser circuit 502. Examples of isolation devices that may be used in embodiments of the present invention are described in M. E. Schafer and P. A. Lewin, “The Influence of Front-End Hardware on Digital Ultrasonic Imaging,” IEEE Trans. Sonics Ultrasonics SU-31, 295-306 (1984), the entire disclosure of which is incorporated herein by reference for all purposes. An amplified received signal 503 produced by the received signal amplifier 500 may be analyzed to position the reflective scatterer, as described in more detail in FIG. 2B.

FIG. 2B illustrates the operation of a transducer as an ultrasound pulse-echo transmitter in accordance with an embodiment of the present invention. In certain embodiments of the present invention, the transducer element 210a may be used as a pulse-echo unit to precisely position the reflective scatterer 400 so that the scattering end 401 is at an optimal location in the HIFU hydrophone 200 relative to the transducer element 210a. In an embodiment of the present invention, a single transducer element may be used that may be substantially curved and/or substantially spherically curved. In such an embodiment, the transducer element 210a may be used as an ultrasound pulse-echo transmitter, as described in more detail below, to locate a center of curvature, which may be a center of spherical curvature if the transducer element 210a is substantially spherically curved, for the transducer element 210a.

In different embodiments, the HIFU hydrophone 200 may comprise two or more transducer elements and the optimal location may be a point wherein acoustic waves reflected and/or scattered from the optimal point may reach the two or more transducer elements so as to be in phase and reinforce each other to provide a combined signal. As such, depending upon the alignment of the transducer elements, the optimal location may be a center of curvature, a spherical center point, a position equidistant from the transducer elements, and/or the like.

To precisely determine the optimum location, the transducer element 210a may be driven by a pulser circuit to transmit a series of acoustic waves 602. The acoustic waves 602 may be reflected by the scattering end 401 as a series of reflected waves 603 that may be reflected onto the transducer element 210a. Transducer element 210a may produce an electrical signal in response to the series of reflective waves 603 incident upon it. This signal may be processed, amplified etc., by a circuit 600 to provide an output 601. In embodiments comprising more than one transducer element, the series of reflected waves 603 may be received by each of the more than one transducers and measured. In the illustrated embodiment, the series of reflected waves 603 may be received by the second transducer element 210b. Transducer element 210a may also produce an electrical signal in response to the series of reflective waves 603 incident upon it, and this signal may be processed by a second circuit 604 to provide an output 605. In certain embodiments, the first circuit 600 and the second circuit 604 may be combined to provide a combined output.

In embodiments of the present invention, the reflective scatterer 400 may be moved using the positioning mechanism 300 while the transducer element 210a and or the transducer element 210b is used as a pulse-echo device. For correct positioning of the scatterer, i.e., when it is positioned at a focal point of the transducer element 210a or at a combined focal point of both the transducer element 210a and the transducer element 210b, the output 601 is maximized. Similarly, in embodiments with more than two transducer elements, when the scatterer end 401 is positioned at the optimal location outputs from each transducer element or a signal combining all of the outputs from each of the transducer elements is maximized. In embodiments of the present invention comprising a plurality of the transducer elements the reflective scatterer 400 may be moved logically between the different transducers to ascertain the optimal location. Merely by way of example, with regard to FIG. 2A, for the transducer elements 210a and 210c, the positioning mechanism 300a may be a primary adjustment mechanism and for the transducer elements 210b and 210d the positioning mechanism 300b may be the primary adjustment mechanism.

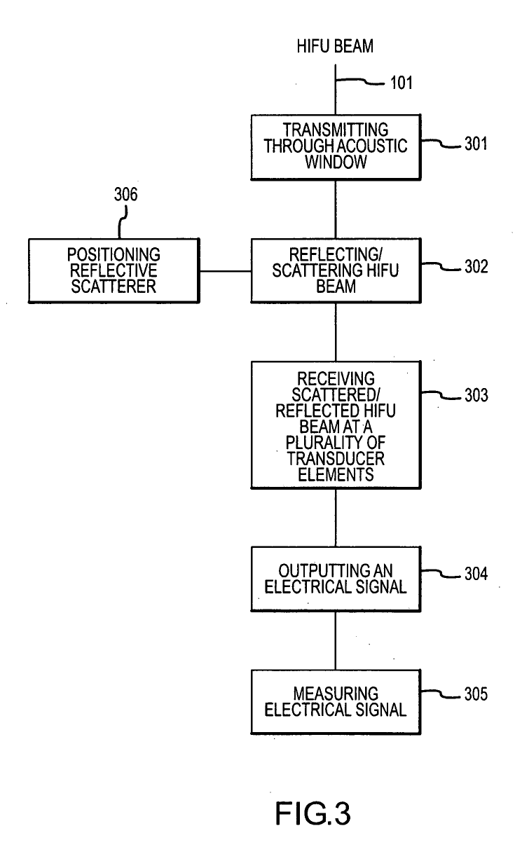

FIG. 3 provides a flow diagram providing an overview of the functional hierarchy used in an embodiment of the present invention. In step 301, a HIFU beam 101 may be transmitted through an acoustic window. In certain aspects the acoustic window may simply be an opening into an interior volume defined be a support structure that may support one or more transducer elements. In other aspects, the acoustic window may comprise a membrane or the like that may be transparent to the HIFU beam 101 and/or may be impermeable. In step 302, the HIFU beam may be aligned to enter the support structure so as to be incident upon and reflected/scattered from a reflective scattering device. In certain aspects, the reflective scattering device may be tapered and/or shape to have an active area that may be smaller in size than the wavelength of the HIFU beam. The reflective scatterer may be positioned so that the HIFU field is incident the active are of the reflective scattering device.

In step 303, the scattered/reflected HIFU beam may be received by a plurality of transducer elements. In an embodiment of the present invention, each transducer element may be substantially spherically curved and the plurality of the transducer elements may be positioned symmetrically around the active area of the reflective scatterer. In step 304, each of the transducer elements may output an electrical signal in proportion to the amount of the reflected/scattered HIFU beam incident upon the transducer element. In step 305, the electrical signals from each of the transducer elements may be combined and processed and an output produced that is proportional to the strength of the HIFU beam. Utilizing the combined and processed output, characteristics of the HIFU beam may be determined and measured. Because of the symmetrical position of the transducer elements, the reflected/scattered HIFU beam may be in phase when it is received by the transducer elements providing an additive effect that may increase the received signal strength and may provide for ease of processing, manipulating and/or processing of the electrical signal.

In a further step 306, positioning of the reflective scatterer may be performed by using the transducer elements as pulse-echo devices and by measuring the echo—the reflected/scattered pulses from the reflective scatterer. In this way, the reflective scatterer may be moved until a maximum echo value is received by the transducer elements establishing that the reflective scatterer is positioned at the point of symmetry of all of the transducer elements.

Having described several embodiments, it will be recognized by those of skill in the art that various modifications, alternative constructions, and equivalents may be used without departing from the spirit of the invention. Accordingly, the above description should not be taken as limiting the scope of the invention, which is defined in the following claims.

1. A self-contained hydrophone for measuring high intensity ultrasonic fields, comprising:
a first impermeable barrier transparent to high intensity ultrasonic fields;
a second impermeable barrier transparent to high intensity ultrasonic fields;
means for supporting comprising an inner surface and two ends, wherein a first end of the means for supporting is coupled with the first impermeable barrier and a second end of the means for supporting is coupled with the second impermeable barrier, and wherein the inner surface, the first impermeable barrier and the second impermeable barrier define an interior sealed volume;
means for transducing acoustic energy to electrical energy, wherein the means for transducing is supported by the inner surface of the means for supporting; and
means for scattering ultrasound wave energy a portion of which is disposed within the interior sealed volume.

2. The self-contained hydrophone for measuring high intensity ultrasonic fields as recited in claim 1, wherein the means for transducing is substantially spherically curved in shape.

3. The self-contained hydrophone for measuring high intensity ultrasonic fields as recited in claim 1, further comprising:
means for reducing cavitation of the scattering means disposed within the interior sealed volume.

4. The self-contained hydrophone for measuring high intensity ultrasonic fields as recited in claim 1, further comprising:
means for driving the means for transducing to produce acoustic energy waves;
means for identifying a strength of an electronic signal produced by the means for transducing in response to reception of the acoustic energy waves; and
means for moving the means for scattering within the interior sealed volume.

5. The self-contained hydrophone for measuring high intensity ultrasonic fields as recited in claim 1, further comprising:
second means for transducing acoustic energy to electrical energy supported on the interior surface, wherein the first means for transducing and the second means for transducing are positioned substantially equidistant from the portion of the means for scattering disposed within the interior sealed volume.

6. The self-contained hydrophone for measuring high intensity ultrasonic fields as recited in claim 5, further comprising:
means for driving the first means for transducing to produce acoustic energy waves;
means for identifying strength of electronic signals produced by the first transducing means and the second transducing means in response to reception of the acoustic energy waves; and
means for moving the scattering means within the interior sealed volume.

7. A self-contained, wide bandwidth hydrophone for measuring high intensity ultrasonic fields, comprising:
a shell structure substantially spherically curved about a center point and defining a sphere with one or more truncated ends, wherein the shell structure has an interior surface defining an interior volume and the one or more truncated ends define at least one opening in the shell structure providing access to the interior volume;
a plurality of transducer elements coupled with the interior surface; and
a reflective scatterer coupled with the shell structure and configured to provide that a part of the reflective scatterer is located at the center point.

8. The self-contained, wide bandwidth hydrophone for measuring high intensity ultrasonic fields as recited in claim 7, wherein each of the plurality of the transducer elements comprise a piezopolymer material.

9. The self-contained, wide bandwidth hydrophone for measuring high intensity ultrasonic fields as recited in claim 7, wherein each of the plurality of the transducer elements comprise polyvinylidene difluoride.

10. The self-contained, wide bandwidth hydrophone for measuring high intensity ultrasonic fields as recited in claim 7, wherein the reflective scatterer comprises a fiber-optic glass fiber.

11. A self-contained, wide bandwidth hydrophone for measuring high intensity ultrasonic fields, comprising:
a shell structure substantially spherically curved about a center point and defining a sphere with two truncated ends;
a first and a second membrane at each of the two truncated ends, wherein the first and the second membranes are transparent to high intensity ultrasonic fields and the shell structure and the acoustically transparent membranes define an interior sealed volume;
two or more transducer elements coupled with an inner surface of the shell structure, wherein each of the two or more transducer elements conform to the substantially spherically curved shape of the inner surface; and
a reflective scatterer coupled with the shell structure, wherein a portion of the reflective scatterer is positioned at the center point.

12. The self-contained, wide bandwidth hydrophone for measuring high intensity ultrasonic fields as recited in claim 11, wherein at least one of the first and second membranes is substantially planar.

13. The self-contained, wide bandwidth hydrophone for measuring high intensity ultrasonic fields as recited in claim 11, wherein the first and second membranes define substantially parallel planes.

14. The self-contained, wide bandwidth hydrophone for measuring high intensity ultrasonic fields as recited in claim 11, wherein the reflective scatterer is tapered in shape and a tapered end of the reflective scatterer comprises the portion of the reflective scatterer positioned at the center point.

15. The self-contained, wide bandwidth hydrophone for measuring high intensity ultrasonic fields as recited in claim 11, further comprising:
a pulse circuit connected to each of the two or more transducer elements and configured to drive each of the two or more transducer elements to produce an acoustic signal:
a reception circuit connected to each of the two or more transducer elements and configured to identify strength of the acoustic signal received by each of the transducer elements; and
a positioning mechanism attached to the reflective scatterer and configured to move the reflective scatterer.

16. The self-contained, wide bandwidth hydrophone for measuring high intensity ultrasonic fields as recited in claim 11, wherein each of the two or more transducer elements comprises a piezopolymer material.

17. The self-contained, wide bandwidth hydrophone for measuring high intensity ultrasonic fields as recited in claim 11, wherein each of the two or more transducer elements comprises polyvinylidene difluoride.

18. The self-contained, wide bandwidth hydrophone for measuring high intensity ultrasonic fields as recited in claim 11, wherein the reflective scatterer comprises a one of a tapered glass rod and a fiber optic glass fiber.

19. The self-contained, wide bandwidth hydrophone for measuring high intensity ultrasonic fields as recited in claim 11, further comprising:
a cavitation mitigating liquid disposed within the interior sealed volume.

20. A method for measuring a high intensity ultrasonic field, comprising:
transmitting the high intensity ultrasonic field onto a reflective scatterer;
scattering and reflecting the high intensity ultrasonic field from a portion of a reflective scatterer;
receiving the scattered and reflected high intensity ultrasonic field at a plurality of transducer elements, wherein the transducer elements are arranged to be equidistant from the portion of the reflective scatterer;
receiving an electrical signal from each of the plurality of transducer elements; and
measuring a combined value of the electrical signals.

21. The method for measuring a high intensity ultrasonic field as recited in claim 20, wherein each of the plurality of transducer elements is substantially spherically curved in shape and the portion of the reflective scatterer is located at a center point of the plurality of transducer elements.

22. The method for measuring high intensity ultrasonic fields as recited in claim 20, further comprising:
driving a one of the plurality of transducer elements with an electric field to produce an acoustic pulse wave;
receiving an output from each of the plurality of the transducer elements in response to the acoustic pulse wave;
combining the outputs
moving the reflective scatterer; and
fixing a position the reflective scatterer when the combined outputs are a maximum.

23. The method for measuring high intensity ultrasonic fields as recited in claim 20, further comprising:
disposing a cavitation mitigating liquid in contact with the portion of the reflective scatterer.

24. The method for measuring high intensity ultrasonic fields as recited in claim 20, further comprising:
disposing a cavitation mitigating liquid in contact with each of the plurality of transducer elements.

25. The method for measuring high intensity ultrasonic fields as recited in claim 20, further comprising:
monitoring resistance change across each of the plurality of transducer elements.

26. A method for providing a self-contained hydrophone for measuring high intensity ultrasonic fields, comprising:
providing a first acoustic window, wherein the first acoustic window is transparent to the high intensity ultrasonic fields;
providing a second acoustic window, wherein the second acoustic window is transparent to the high intensity ultrasonic fields;
providing a shell structure wherein the shell structure is spherically curved about a center point and defines a sphere with a first truncated end and a second truncated end;
coupling the first acoustic window with the first truncated end and coupling the second acoustic window with the second truncated end, wherein the shell structure and the first and the second acoustic window define an interior sealed volume;
coupling a first transducer element to an interior surface of the shell structure inside the interior sealed volume, wherein the transducer element is substantially spherically curved in shape around the center point; and
positioning an end of a reflective scatterer within the interior sealed volume at the center point.

27. The method for providing the self-contained hydrophone for measuring high intensity ultrasonic fields as recited in claim 26, further comprising:
providing a driving circuit configured to drive the transducer element to produce acoustic waves; and
providing a receiving circuit configured to provide an output corresponding to strength of the acoustic waves received by the transducer element.

28. The method for providing the self-contained hydrophone for measuring high intensity ultrasonic fields as recited in claim 26, further comprising:
filling the interior sealed volume with a cavitation mitigating liquid.
